(19) United States
(12) Patent Application Publication
(10) Pub. No.: US 2006/0280691 A1
(43) Pub. Date: Dec. 14, 2006

(54) Spray freeze dried liposomal ciprofloxacin powder aerosol drug delivery

(75) Inventors: Zhaolin Wang, Richmond, VA (US); Helena Orszanska, Edmonton (CA); Warren H. Finlay, Edmonton (CA)

Correspondence Address: OGILVY RENAULT LLP, 1981 MCGILL COLLEGE AVENUE, SUITE 1600, MONTREAL, QC, H3A2Y3, CA

(73) Assignee: University of Alberta

(21) Appl. No.: 11/445,347

(22) Filed: Jun. 2, 2006

Related U.S. Application Data
(60) Provisional application No. 60/689,968, filed on Jun. 13, 2005.

(51) Int. Cl. A61K 9/127; A61K 9/14; A61L 9/04

(52) U.S. Cl. 424/46; 424/450

(57) ABSTRACT

A powder for inhalatory aerosol delivery, the powder having: spray freeze dried liposome particles with a biologically active agent, such as an antibiotic, encapsulated within a phospholipid, and a method of producing a powder for inhalatory aerosol delivery, the method including the steps of: mixing a biologically active agent with a phospholipid to form a liquid liposome suspension; and spray freeze drying the liposome suspension to form particles of powder.

### CROSS-REFERENCE TO RELATED APPLICATION

This application claims priority from provisional U.S. application Ser. No. 60/689,968 filed Jun. 3, 2005.

### TECHNICAL FIELD

The invention relates to a powder formulation for inhaled aerosol drug delivery of liposomes that has been prepared using spray-freeze drying.

### BACKGROUND OF THE ART

Inhalation drug delivery is an effective pathway to treat many topical and some systemic illnesses (for example: Finlay W H. 2001. The mechanics of inhaled pharmaceutical aerosols, An introduction. London: Academic Press). Indeed, the number and type of therapeutic agents utilized in inhalation treatment is increasing yearly; an example being the peptides and proteins produced through biotechnology for pulmonary delivery (Adjei A L, Gupta P K. 1998. Inhalation delivery of therapeutic peptides and proteins. New York: Marcel Decker)

One particular type of drug delivery system that has been explored with a number of antimicrobial and anticancer drugs is liposomal encapsulation of the active component (for example: Oh Y K, Nix D E, Straubinger R M. 1995. Formulation and efficacy of liposome-encapsulated antibiotics for therapy of intracellular mycobacterium avium infection. Antimicr Agts Chemo 39: 2104-2111). Generally, liposomes are described as artificial microscopic vesicles having a core usually of an aqueous active agent enclosed within one or more phospholipid layers. In successful circumstances, this enclosure in phospholipid alters the pharmacokinetics to an extent that active agent drug retention time is increased and drug toxicity is reduced, thereby prolonging the half life of the drug in the body.

Typically, liposomal formulations have been delivered by nebulization, where liquid formulations are atomized. Concerns arise when nebulizers are used to deliver a liposomally encapsulated agent from drug stability and leakage perspectives (Taylor K M G, Taylor G, Kellaway I W, Stevens J. 1990. The stability of liposomes to nebulisation. Int J Pharm 58: 57-61). To circumvent these issues, dry powder formulations have been examined. Desai et al. (Desai T R, Wong J P, Hancock R E W, Finlay W H. 2002. A novel approach to the pulmonary delivery of liposomes in dry powder form to eliminate the deleterious effect of milling. J Pharm Sci 91 (2): 482-491) examined the effects of lyophilization and jet milling on the efficacy-of a liposomal formulation and found significant leakage due to stresses induced in the separate drying and milling processes.

Desai et al proposed a solution that utilized spontaneous production of liposomes. It is known that, due to electrostatic interactions, phospholipids will spontaneously encapsulate a particle of suitable charge in an ionic solution, thereby creating a liposomal particle. This process generated promising in vitro results, however significant losses were encountered in the milling process and observed suboptimal dispersion due to the auto-adhesive properties of the powder.

Features that distinguish the present invention from the background art will be apparent from review of the disclosure, drawings and description of the invention presented below.

### DISCLOSURE OF THE INVENTION

The invention provides a powder for inhalatory aerosol delivery, the powder having: spray freeze dried liposome particles with a biologically active agent, such as an antibiotic, encapsulated within a phospholipid.

The invention also provides a method of producing a powder for inhalatory aerosol delivery, the method including the steps of: mixing a biologically active agent with a phospholipid to form a liquid liposome suspension; and spray freeze drying the liposome suspension to form particles of powder.

The inventors examined the properties of a spray freeze dried agent that addresses many of the problems encountered by previous formulations and manufacturing methods. The Example described herein relates to a spray freeze dried ciprofloxacin formulation that relies on the spontaneous formation of liposomes. Ciprofloxacin was selected in the Example as the active agent because it is a broad spectrum antibiotic and demonstrates sustained release and acts as an effective therapeutic agent against Francisella tularensis infection when delivered liposomally (Wong J P, Yang H, Blasetti K L, Schnell G, Conley J, Schofield L N. 2003. Liposome delivery of ciprofloxacin against intracellular Francisella tularensis infection. J Contr Rel 92: 265-273).

Spray freeze drying produces stable formulations with superior aerodynamic and dissolution properties compared to other dry powder manufacturing methods (Maa Y F, Nguyen P A, Sweeney T, Shire S J, Hsu C C. 1999. Protein inhalation powders: spray drying v. spray freeze drying. Pharm Res 16: 249-254).

The invention provides a spray freeze dried manufacturing method for a novel formulation. The results of a reconstitution study, and the dispersion properties of the aerosol using a passive inhaler are described below.

### DESCRIPTION OF THE DRAWINGS

In order that the invention may be readily understood, one embodiment of the invention is described and illustrated by way of an Example in the accompanying drawings.

FIG. 1 is a chart plotting the encapsulation efficiencies of the Example suspension before spray freeze drying and the encapsulation efficiencies of the Example powder reconstituted in various liquid media.

FIG. 2 is a chart plotting encapsulation efficiencies of the Example powder reconstituted in various liquid media at a dilution fivefold times that of FIG. 1.

FIG. 3 is a representative Scanning Electron Microscope image showing the morphology of a particle from the Example powder.

Further details of the invention and its advantages will be apparent from the detailed description included below.

### DETAILED DESCRIPTION OF PREFERRED EMBODIMENTS

An Example formulation described below contains phospholipid, (namely: Dimyristoyl-sn-glycero-3-phosphoglycerol (DMPG) provided as a sodium salt by Genzyme Pharmaceuticals, Cambridge, Mass., U.S.A.), lactose (Pharmatose 325M, DMV International, Veghel, The Netherlands) and ciprofloxacin (US Biological, Swampscott, Mass., U.S.A.) as an Example drug in a weight percent ratio 5:17:1, respectively. The formulation forms a smooth suspension upon vortexing (2×30 sec—4×30 sec in 1 hr) and remains stable at 4° C. for several days.

Spray freeze drying was performed with a two-fluid nozzle (Spray System Co., Wheaton, Ill., U.S.A.), in which compressed nitrogen and a peristaltic pump (Chem Tech, Punta Gorda, Fla., U.S.A.) were used to drive the formulation. The suspension was atomized into a flask containing liquid nitrogen. Following atomization, the remaining liquid nitrogen was allowed to evaporate, and the resulting powder was dried for 48 hrs in a freeze drier (Labconco Corp., Kansas City, Mo., U.S.A.). The powder was subsequently collected and stored in a sealed vial at 4° C.

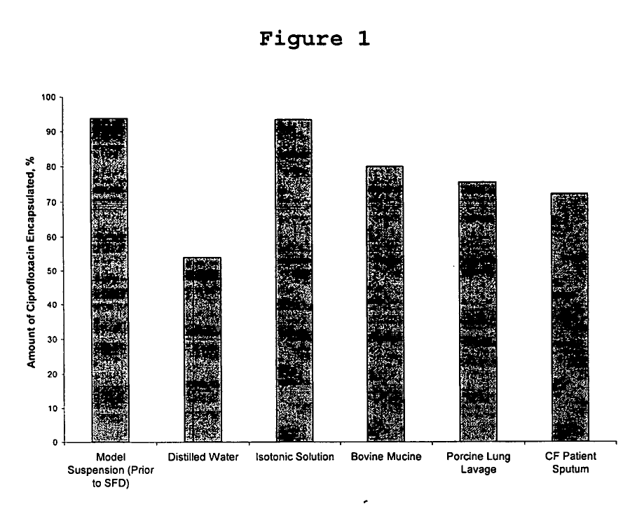

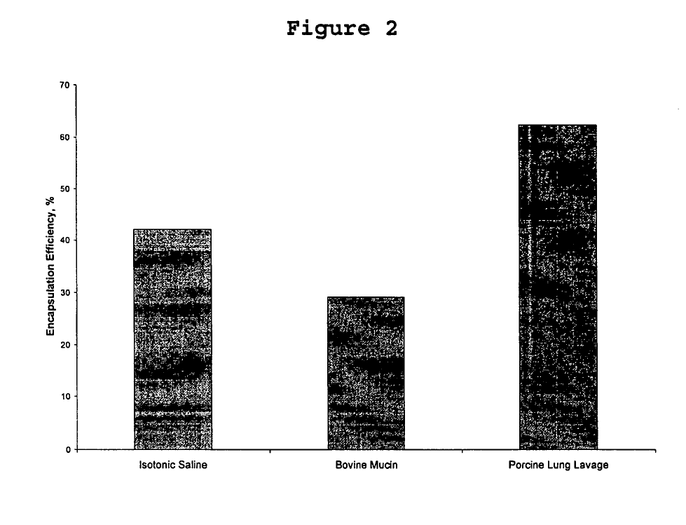

In a Vitro Reconstitution process, the ability of the phospholipids to encapsulate ciprofloxacin was tested in various liquid media. Prior to spray freeze drying, the suspension of lactose, DMPG and ciprofloxacin was tested for leaking of the active agent ciprofloxacin. The suspension was centrifuged at 4° C. and 14 000 rpm for 1 hr (Allegra 21R, Beckman Coulter, Fullerton, Calif., U.S.A.). The supernatant and pellet were collected and dissolved separately in methanol. The resulting solutions were analyzed for ciprofloxacin content using UV spectroscopy (UV absorbance at λ=278 nm, Diode Array Spectrophotometer, model 8452A, Hewlet Packard, Tulsa, Okla., U.S.A.). Subsequent to spray freeze drying the powder was reconstituted in various liquid media, including distilled water, isotonic saline solution, bovine mucin type 1 from submaxillary glands (Sigma-Aldrich, St. Louis, Mo., U.S.A.), porcine mucin (extracted post-mortem using lung lavage), and ex vivo human cystic fibrosis patient sputum (spontaneous expectoration with dental cotton packing between cheeks and gums to minimize admixture with saliva) diluted fivefold with isotonic saline (results shown in FIG. 1 described below). The identical procedure utilized to analyze encapsulation in the Example drug suspension was used to evaluate leaking in the reconstituted powders (results shown in FIG. 1 also described below). Leaking was also evaluated in the aforementioned liquids diluted fivefold with distilled water (results shown in FIG. 2 described below).

The resultant spray freeze dried powder had the following properties. The fine particle fraction (FPF) and mass median aerodynamic diameter of the powder was measured using a Mark II Anderson Cascade Impactor (Graseby Anderson, Smyma, Ga., U.S.A.) with cut points recalibrated at 60 l/min. Deagglomeration and powder delivery was achieved with a proprietary passive dry powder inhaler. The flow rate of the inhaled air/particle mixture was monitored with a pneumotachometer (PT 4719, Hans Rudolph Inc., Mo., U.S.A.). Prior to testing, the impactor plates were sprayed with a release agent (316 Silicone Release Spray, Dow Corning, Midland, Mich., U.S.A.). Each impactor plate was washed with 5 ml of CHCl₃/MeOH/H₂O in a volume ratio of 1.3/2.6/1.1. The lactose was separated from the remaining ingredients using the method described in Bligh et al (Bligh E G, Dyer W J. 1959. A rapid method of total lipid extraction and purification. Cdn J Biochem Phys 37: 911-917).

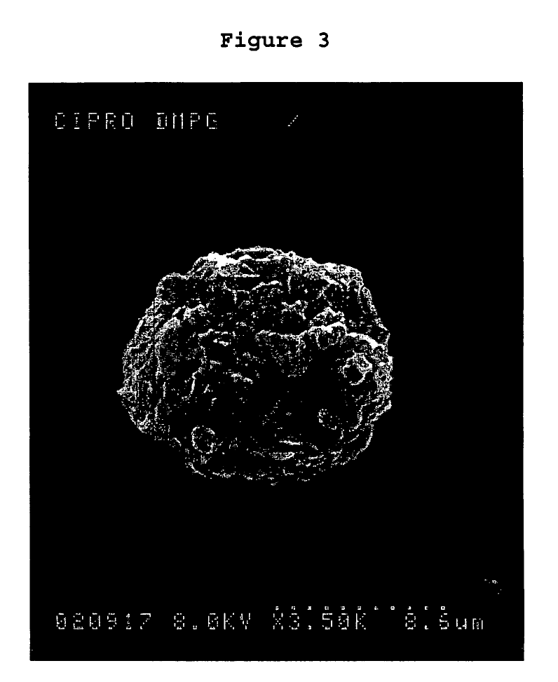

The preseparator was washed with 10 ml MeOH and the inhaler with 10 mL MeOH/H₂O, in a volume ratio of 4:1. The resulting solutions were analyzed for ciprofloxacin content using UV spectroscopy following the previously stated procedure and equipment. Upon powder reconstitution, liposome particle size was measured using photon correlation spectroscopy (Malvern Zetasizer 3000, Malvern Instruments, UK). Powder morphology was analyzed with electron microscopy (shown in FIG. 3).

Airway Deposition and Surface Fluid Simulation were modelled using a numerical lung deposition model coupled with an airway surface liquid (ASL) model (Lange C F, Hancock R E W, Samuel J, Finlay W H. 2001. In vitro aerosol delivery and regional airway surface liquid concentration of a liposomal cationic peptide, J Pharm Sci 90:1647-1657) was utilized to predict ciprofloxacin concentration in the tracheobronchial generations of normal lungs. The numerical lung deposition model utilized an inhalation flow rate of 60 l/min with a mucous production rate of 10 ml/day and a tracheal velocity of 10 mm/min, as well as the measured mass median aerodynamic diameter and geometric standard deviation of the powder.

The example showed experimental results where bovine mucin, porcine lung lavage and fivefold diluted cystic fibrosis sputum demonstrated encapsulation efficiencies greater than 70% as shown in FIG. 1.

The spray freeze dried particles were reconstituted in various fluids. The reconstituted encapsulation efficiency of the Example drug in water was the lowest of all the fluids tested. This is readily explained through the results of (Crowell K J, Macdonald P M. 1999. Surface charge response of the phosphatidylcholine head group in bilayered micelles from phosphorus and deuterium nuclear magnetic resonance. Biochimica et Biophysica Acta. 1416: 21-30) who found that phospholipid DMPG formulations require an ionic solution for autoassembly of lipids.

The highest reconstituted encapsulation efficiency was observed in isotonic saline. Drawing comparison to the three pulmonary liquids, it is possible that the presence of one or more pulmonary surfactants slightly decreases the amount of encapsulation. Dilution of the liquid samples fivefold also caused a reduction in encapsulation efficiency, as shown in FIG. 2. This is a known and expected result as encapsulation depends on concentration of lipoplexes, ionic strength and the presence of surfactants.

The particles of the Example powder formulation demonstrated high specific surface area characteristic of so-called engineered powders. A representative Scanning Electron Microscope image of a powder particle is shown in FIG. 3.

The improved physical properties of the powder particles, most importantly mass median aerodynamic diameter, were apparent in deposition testing. An exceptional mass median aerodynamic diameter was observed using a proprietary dry powder inhaler with a cascade impactor at a flow rate of 60 l/min. This result demonstrates the strong deagglomeration capabilities coupled with the favorable aerodynamic properties of the powder. Utilizing this powder formulation with an advanced inhaler reduces the amount of mouth-throat deposition and increases lung deposition compared with prior art aerosol formulations of liposomal ciprofloxacin.

The average mass median aerodynamic diameter was found to be 2.8 μm (SD 1.0 μm), while the fine particle fraction was calculated to be 60.6% (SD 12.2%). The average mass of ciprofloxacin in the fine particle fraction per mass of power was determined to be 20.6 μg ciprofloxacin/mg of powder (SD 5.6 μg/mg).

Liposome particle size was measured after powder reconstitution in the saline solution. A mean volume analysis showed that 91% of the particles had a diameter smaller than 600 nm.

The above described Example demonstates the following general conclusions. Electrostatic properties of phospholipids, such as DMPG, and ciprofloxacin allow the spontaneous production of liposomally encapsulated ciprofloxacin particles in an ionic aqueous media. Ciprofloxacin is also known for electrostatic interactions with phosphatidylglycerols and zwitterionic phospholipids. Indeed, in vitro evidence suggests that these particles will auto-assemble in various liquid media, with encapsulation efficiency depending on surfactant, lipid concentration and ionic strength.

Utilizing this property in a powder formulation circumvents many of the problems associated with delivering liposomal particles to the respiratory tract. Fluid droplets created in nebulizers experience shear, as well as shock waves and kinematic discontinuities that impose destructive forces on the particles. Lyophilization and jet milling also lead to deleterious effects on the particles. Formation of liposomal particles in vivo removes the sensitive liposomal particles from the manufacturing and delivery stages of aerosol delivery.

In the Example ciprofloxacin was used as an example active agent, however the method is applicable to many other agents. Ciprofloxacin has the unusual property of having a solubility limit increased almost two orders of magnitude when liposomally encapsulated (Maurer N, Wong K F, Hope M J, Cullis P R. 1998. Anomalous solubility behavior of the antibiotic ciprofloxacin encapsulated in liposomes: a H-NMR study. Biochimica et Biophysica Acta 1374: 9-20), which is beneficial from a manufacturing perspective. Spray freeze drying produces high quality powders, but requires significantly increased time and energy input due to the reduced heat and mass transfer rates associated with a frozen medium in the drying phase (supra, Maa and Prestrelski, 2000). The liposomal encapsulation efficiency of ciprofloxacin tested before spray freeze drying in this study was 93.8%, which suggests an increase in ciprofloxacin solubility. The formulation prior to freezing may benefit from reduced water content, for example approximately two orders of magnitude less water, compared to a non-liposomally-encapsulated ciprofloxacin solution. Consequently, an increase in manufacturing efficiency can be realized through liposomal encapsulation of ciprofloxacin, due to the reduced water content and therefore reduced time and energy used in the drying process.

Reconstitution of the Example powder in various representative pulmonary fluids demonstrated promising liposomal encapsulation. The modeled experimental results indicate that actual in vivo encapsulation does occur. Upon administration of a 20 mg powder dosage (the approximate maximum a patient can inhale without coughing), the Example deposition simulation predicts a minimum ciprofloxacin concentration of 5 mg/l, occurring in the most distal tracheobronchial generation. This concentration is above the minimum inhibitory concentration (MIC) of many bacteria causing respiratory infection, including Pseudomonas aeruginosa (MIC90 4 mg/l), Streptococcus pyogenes (MIC90 1 mg/l), Neisseria gonorrhoeae (MIC90 0.004 mg/l), Bacillus anthracis (MIC 1.6 mg/l), and many other aerobes (Zhanel G G, Ennis K, Vercaigne L, Walkty A, Gin A, Embil J, Smith H, Hoban D J. 2002. A critical review of the fluoroquinolones: Focus on respiratory tract infections. Drugs 62(1): 13-59 and, Brook J. 2002. The prophylaxis and treatment of anthrax, Int J Antimicrobial Agents. 20: 320-325). The present powder formulation delivery may thus be a possible treatment pathway for numerous bacterial infections.

Described above are the characteristics and results of a Example drug that relies on the spray freeze drying process along with the spontaneous formation of liposomes in ionic aqueous media. The Example drug demonstrated high encapsulation efficiency in three characteristic pulmonary fluids. The powder has advantageous stability, aerodynamic and dissolution properties due to the spray freeze dried process, requires fewer manufacturing steps, and is less adhesive than its jet-milled counterpart.

The above Example describes a powder formulation for inhaled aerosol drug delivery of liposomes prepared using spray-freeze drying. Aerosol dispersion properties of this formulation were assessed using a new passive inhaler, in which the powder was entrained at a flow rate of 60 l/min. A mass median diameter of 2.8 μm was achieved for this Example formulation with ciprofloxacin as the active agent drug. The reconstitution of the powder in various aqueous media gave drug encapsulation efficiencies as follows: 50% in water, 93.5% in isotonic saline, 80% in bovine mucin, 75% in porcine mucus and 73% in fivefold diluted ex vivo human cystic fibrosis patient sputum. Airway surface fluid concentrations predicted by simulation upon inhaled aerosol delivery are above typical minimum inhibitory values, indicating the favorability of the present formulation and delivery.

The invention encompasses a spray freeze dried powder of liposomes composed of: a phospholipid; and an active agent, such as ciprofloxacin, in a weight percent ratio of about 5:1. The liposomes may include a carrier, such as lactose, with a ratio of phospholipid: carrier: active agent in a weight percent ratio about 5:17:1, respectively. The invention provides particles with a average mass median aerodynamic diameter of the spray freeze dried particles within the range of 2.0-4.0 μm, and the fine particle fraction of the powder is within the range of 45%-75%. The average mass of active agent, such as ciprofloxacin, in the fine particle fraction per mass of powder is within the range of about 14-26 μg active agent/mg of spray freeze dried powder. A range of between 80%-100% of the particles provided by the powder of the invention have a diameter smaller than 600 nm. Reconstitution of the powder in vivo will result in drug encapsulation efficiencies within the range of 70%-95% based on the experiment described herein.

Although the above description relates to a specific preferred embodiment as presently contemplated by the inventors, it will be understood that the invention in its broad aspect includes mechanical and functional equivalents of the elements described herein.

We claim:

1. A powder for inhalatory aerosol delivery, the powder comprising:
spray freeze dried liposome particles having a biologically active agent encapsulated within a phospholipid.

2. A powder according to claim 1 wherein the weight percent ratio of phospholipid: active agent is about 5:1.

3. A powder according to claim 1 wherein the liposome includes a carrier.

4. A powder according to claim 1 the weight percent ratio of phospholipid: carrier: active agent is about 5:17:1.

5. A powder according to claim 1 the active agent is an antibiotic.

6. A powder according to claim 5 the active agent is ciprofloxacin.

7. A powder according to claim 3 the carrier is lactose.

8. A powder according to claim 1 wherein an average mass median aerodynamic diameter of the spray freeze dried particles within the range of 2.0-4.0 μm.

9. A powder according to claim 1 wherein a fine particle fraction of the powder is within the range of 45%-75%.

10. A powder according to claim 1 wherein an average mass of active agent in a fine particle fraction per mass of powder is within the range of about 14-26 μg active agent per mg of spray freeze dried powder.

11. A powder according to claim 1 wherein between 80%-100% of the particles have a diameter smaller than 600 nm.

12. A powder according to claim 1 wherein a reconstitution of the spray freeze dried particles in a liquid results in drug encapsulation efficiencies within the range of 70%-95%.

13. A powder according to claim 12 wherein the liquid is selected from the group consisting of: in vivo human pulmonary mucus; isotonic solution; bovine mucine; porcine lung lavage; and diluted human sputum.

14. A powder according to claim 1 wherein the liposome particles before spray freeze drying comprise liposomes formed within a liquid suspension having an encapsulation efficiency within the range of 90%-95%.

15. A method of producing a powder for inhalatory aerosol delivery, the method comprising:
mixing a biologically active agent with a phospholipid to form a liquid liposome suspension; and
spray freeze drying said liposome suspension to form particles of said powder.

16. A method according to claim 15 wherein the suspension is sprayed through an atomizing nozzle into liquid nitrogen.

17. A method according to claim 16 wherein particles are separated from said liquid nitrogen after spraying.

18. A method according to claim 17 wherein particles are dried in a freeze dryer after being separated from the liquid nitrogen.

19. A method according to claim 15 wherein the spray freeze dried particles are reconstituted in a liquid resulting in drug encapsulation efficiencies within the range of 70%-95%.

20. A method according to claim 19 wherein the liquid is selected from the group consisting of: in vivo human pulmonary mucus; isotonic solution; bovine mucine; porcine lung lavage; and diluted human sputum.
